(19) United States
(12) Patent Application Publication
(10) Pub. No.: US 2021/0127976 A1
(43) Pub. Date: May 6, 2021

(54) SYSTEM AND METHOD FOR ASSESSING PREPAREDNESS FOR IMAGING PROCEDURES

(71) Applicant: KONINKLIJKE PHILIPS N.V., EINDHOVEN (NL)

(72) Inventors: Olga STAROBINETS, CAMBRIDGE, MA (US); Sandeep Madhukar DALAL, WINCHESTER, MA (US); Ranjith Naveen TELLIS, CAMBRIDGE, MA (US); Hareesh CHAMARTHI, CAMBRIDGE, MA (US); Yuechen QIAN, LEXINGTON, MA (US); Vijay PARTHASARATHY, LEXINGTON, MA (US)

(21) Appl. No.: 17/084,781

(22) Filed: Oct. 30, 2020

Related U.S. Application Data
(60) Provisional application No. 62/929,121, filed on Nov. 1, 2019.

(51) Int. Cl. A61B 5/00; G16H 10/60; G16H 15/00; G16H 50/30; G06F 40/58; G01R 33/28; G01R 33/56; A61B 5/11; A61B 5/055

(52) U.S. Cl. CPC A61B 5/0033; G16H 10/60; G16H 15/00; G16H 50/30; A61B 5/055; G01R 33/288; G01R 33/5601; A61B 5/1112; A61B 5/7475; G06F 40/58

(57) ABSTRACT

An apparatus (10) includes at least one electronic processor (20) programmed to: receive scheduled imaging examination information (13) for a medical imaging examination which the patient is to undergo including at least a patient identifier, a scheduled date of the medical imaging examination, and an imaging modality of the medical imaging examination; extract patient information (29) from one or more medical records of the patient based on the scheduled imaging examination information; and generate one or more preparatory action items (15) to be performed in preparation for the medical imaging examination based on the patient information and the scheduled imaging examination information.

### CROSS REFERENCE TO RELATED APPLICATIONS

This application claims the benefit of U.S. Provisional Patent Application No. 62/929,121 filed Nov. 1, 2019. These applications are hereby incorporated by reference herein.

### FIELD

The following relates generally to the medical imaging arts, medical imaging workflow, imaging procedure patient preparation, and related arts.

### BACKGROUND

Medical imaging is in high demand. As the world population ages, the demand for efficient, safe, high quality imaging may continue to grow, putting further pressure on medical imaging centers and their staff. In order to image patients quickly and safely, while maintaining high throughput and quality standards, an imaging provider has to establish an efficient workflow.

The imaging workflow typically begins with scheduling the patient for an imaging examination. This establishes the date and imaging modality of the upcoming imaging examination. The scheduled imaging examination is also associated with a patient identifier, that is, a unique identifier of the patient such as a hospital-assigned patient identification number (PID), the patient's social security number (applicable in the United States), the patient's name, and/or so forth. At the time of scheduling, the patient may be informed of various preparatory actions the patient needs to perform prior to the examination, such as obtaining preparatory blood work, fasting for some time interval prior to the examination, and/or so forth. The particular preparatory actions (if any) depend on the imaging modality, the anatomy being imaged, the clinical information that is to be provided by the imaging examination, or so forth.

Other preparatory actions may need to be performed by medical personnel. For example, if the patient does not speak the native language well, then a translator may be needed. In the case of a magnetic resonance imaging (MRI) examination of a patient having a medical implant (e.g., an implanted cardiac maker), a specific MRI scanner may need to be assigned (e.g., one using a lower magnetic field) or a cardiac team may need to be available on standby in case the patient experiences cardiac issues during the MRI examination. If the imaging examination will employ a contrast agent or a radiopharmaceutical, then medical personnel may need to ensure a sufficient quantity of the contrast agent or radiopharmaceutical is available at the time of the imaging examination.

On the day of the imaging examination, the patient checks in with the imaging laboratory, and is typically placed into a waiting room until called for the examination. The examination workflow then continues with a setup phase in which the patient is administered the radiopharmaceutical if applicable, transferred onto the couch or other patient support, and ancillary components such as local imaging coil(s) in the case of MRI secured with the patient. In addition, patients can fill out a screening form on arrival at the imaging facility detailing possible scan contraindications. A technologist then reviews the form with the patient to ensure there are no safety concerns. At this point, allergies, prior scans, implants, etc. are usually communicated and brought to the attention of the medical staff. If such issues are uncovered at this point, there's little that can be done, making delays and cancelations unavoidable.

The actual imaging data acquisition process is then performed: the patient is loaded into the imaging scanner, scheduled imaging sequences are performed, image quality is verified, and the final clinical images are stored to a Picture and Archiving and Communication System (PACS), Cardiovascular Information System (CVIS), or other imaging examinations storage system. The patient is then transferred off the patient support and may be moved to a post-examination waiting room, any requisite paperwork is completed, and the patient is discharged.

Efficient and clinically useful medical imaging depends upon adherence to the established workflow. Medical imaging laboratories typically have at least partially automated workflows encompassing the scheduling process and the workflow commencing with patient check-in through patient discharge from the imaging laboratory. However, it is recognized herein that there are deficiencies in the “preparedness” phase of the workflow, extending between the scheduling and the check-in phases. This phase typically relies upon individual initiative, possibly augmented by occasional electronic reminder emails or text messages. Even when supported by electronic reminders, these usually rely upon individual initiative to set up the reminders during the scheduling phase. Yet, inadequacies in this preparedness phase can lead to delay or cancellation of a medical imaging examination, can reduce clinical value of the acquired medical images, and can even lead to potential harm to the patient.

The following discloses certain improvements to overcome these problems and others.

### SUMMARY

In one aspect, an apparatus includes at least one electronic processor programmed to: receive scheduled imaging examination information for a medical imaging examination which the patient is to undergo including at least a patient identifier, a scheduled date of the medical imaging examination, and an imaging modality of the medical imaging examination; extract patient information from one or more medical records of the patient based on the scheduled imaging examination information; and generate one or more preparatory action items to be performed in preparation for the medical imaging examination based on the patient information and the scheduled imaging examination information.

In another aspect, a non-transitory computer readable medium stores instructions executable by at least one electronic processor to perform a method of generating one or more preparation action items to be performed in preparation for a medical examination which a patient is to undergo. The method includes: receiving scheduled examination information for the medical examination including at least a patient identifier, a scheduled date of the medical examination, and an imaging modality of the medical imaging examination; extracting patient information from one or more medical records of the patient based on the scheduled examination information; generating one or more preparatory action items to be performed in preparation for the medical examination based on the patient information and the scheduled examination information; transmitting one or more reminders to the patient regarding the one or more action items; and automatically updating the one or more action items based on responses from the patient to the one or more reminders.

In another aspect, a method of generating one or more preparation action items to be performed in preparation for a medical examination which a patient is to undergo includes: receiving scheduled imaging examination information for the medical imaging examination including at least a patient identifier, a scheduled date of the medical examination, and an imaging modality of the medical examination; extracting patient information from one or more medical records of the patient based on the scheduled examination information; generating one or more preparatory action items to be performed in preparation for the medical imaging examination based on the patient information and the scheduled examination information; receiving time change information including one or more of a location of the patient, a movement of the patient, a weather forecast, and roadwork; and predict changes to the one or more action items based on the time change information.

One advantage resides in providing an automated workflow tool for automatically identifying, and optionally monitoring and confirming completion of, preparatory action items to be performed for a given medical imaging examination prior to the examination being performed.

Another advantage resides in identifying a preparatory action item comprising patient assessment for presence of a foreign object in the patient before an imaging examination.

Another advantage resides in identifying a preparatory action item comprising providing translation services for a patient before a medical imaging examination.

Another advantage resides in determining a location and/or possible time delays of a patient before a medical imaging examination.

Another advantage resides in identifying a preparatory action item comprising providing a wheelchair or other assistive device to be used by a patient before a medical imaging examination.

Another advantage resides in retrieving prior imaging sessions of a patient before a medical examination is performed for the patient.

Another advantage resides in determining pre-medical examination steps and patient readiness that must be performed before a medical examination.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for assessing preparedness for medical procedures in accordance with the present disclosure.

FIG. 2 shows exemplary operations in a flow chart for use by the apparatus of FIG. 1.

FIG. 3 shows an example assessing preparedness for a medical procedure displayed on the apparatus of FIG. 1.

### DETAILED DESCRIPTION

Some common workflow disruptions caused by inadequacies of the preparedness phase include patients running late or not showing up for their scheduled exams, patients arriving unprepared for the scan, patient requiring translation services with no one available to perform the service, patients reporting a history of an implanted medical device of unknown manufacturer and safety requirements, patients reporting a past contrast allergy of unknown nature, patients recalling a prior scan done at a different facility, etc. These are examples of some common events that, if uncovered at the time of the scan, may derail not only the current exams but delay the following exams, potentially disrupting the entire day. With proper preparation (i.e., by assessing patient exam readiness ahead of time), many of these disruptions can be mitigated or avoided entirely. Unfortunately, a time investment connected to reviewing each patient's history is not negligible.

Moreover, it is not always apparent which medical professional is responsible for recognizing a particular preparatory action item and taking appropriate action. For example, the patient' doctor may schedule an MRI examination without noting that the patient has a cardiac pacemaker. Clerical staff scheduling the medical examination, in turn, may not be aware the patient has a pacemaker; or if aware of the pacemaker may assume that the doctor would have it taken it into account when writing up the examination order. Indeed, some preparatory action items may not be immediately apparent to anyone in the preparedness workflow chain. For example, a patient who is to undergo an MRI and is a retired metalworker (e.g. retired welder, sheet metal worker, or the like) should have a pre-examination CT ocular screening for metal fragments in the patient's eyes. However, identifying this preparatory action item requires both knowledge of the patient's former job and insight to realize that this former job should trigger the CT ocular screening. Identification of other preparatory action items may be obscured by lack of information. For example, a patient who does not speak the native language fluently may be assisted in scheduling the medical imaging examination by an adult son or daughter, so that the patient does not actually speak with the person scheduling the examination. Hence, the scheduler does not know of the patient's need for a translator to be available during the imaging examination.

A successful medical imaging examination depends upon appropriate preparation. For example, an MR compatible pacemaker needs to be accommodated by using a low-strength MRI and/or providing standby cardiac care support; an ocular CT screening is required prior to MRI in the case of an at-risk patient such as a welder; a patient with an allergy to a contrast agent must be administered a different contrast agent and/or be examined using an alternative imaging modality; pre-examination blood work may need to be performed; a sedative may need to be available in the case of a claustrophobic patient; a translator may need to be provided for a patient who does not speak English; and so forth. Failure to perform such preparation can lead to delay or cancellation of the imaging examination. Delays can also be introduced if the patient arrives late for any reason. In existing approaches, preparation is typically handled manually, and often on an ad hoc basis, which can lead to missing key preparatory actions.

The following discloses an improved automated medical imaging workflow system for identification and notification of preparatory activities, and for ensuring the patient arrives on time or providing timely notice of any expected patient delay. When a medical imaging examination is scheduled, the disclosed system accesses the patient's electronic records using the Patient Medical Record Number (PMRN) or other patient identifier, and applies predefined searches to detect relevant patient information. For example, the patient records may be searched for “pacemaker”, “MR conditional” or similar content, or for specific implant make/model information, that indicates the patient has a medical implant. The patient's occupation may be searched, for example using the search pattern: ((welder OR metal) NEAR3 occupation), where in this search pattern “NEAR3 identifies occurrences of “welder” or “metal” within three words of “occupation”. Similar searches can be performed to detect indications of claustrophobia or other relevant patient conditions. Patient hindrances such as language barriers or mobility issues can also be derived from the patient records.

In some embodiments disclosed herein, the scheduled examination information is received, and by combining this with the detected patient information various preparatory action items can be generated. For example, detection that the patient is a “welder” together with the examination being by MRI indicates a pre-examination ocular CT screening should be performed. Detection of an MR conditional medical implant together with the examination being by MRI indicates further preparatory action items. The scheduled examination information by itself may lead to generation of action items such as required fasting, availability of a contrast agent, or so forth.

In other embodiments disclosed herein, the action items are then generated. These may include, for example: scheduling required pre-examination bloodwork; scheduling availability of a cardiac team at the time of the medical imaging examination in the case of a patient with an MR-conditional pacemaker; scheduling use of an MRI with a lower magnetic strength in the case of a patient with an MR-conditional pacemaker; scheduling a different type of imaging modality if the originally scheduled imaging procedure cannot be performed on the patient due to an identified contravening medical condition; and so forth. The action items may be pushed, for example by auto-scheduling blood work or displaying some or all action items on a display used by the person scheduling the imaging procedure. The action items may also be pulled, for example, medical or clerical personnel can retrieve the list of action items.

In some embodiments disclosed herein, status information for each action item is maintained. Hence, for example, if the hematology lab electronic records system logs the patient blood draw, or the blood test results, the action item for pre-exam bloodwork can be automatically updated or removed based on this information. The system may send reminders to the patient (e.g., a text message reminding the patient to “Start fasting in three hours”) and update based on patient responses (e.g., based on receiving a patient acknowledgement of the text message). Alerts may also be sent to the medical imaging laboratory, and/or the patient's doctor or other appropriate medical personnel, if a preparatory action item is still outstanding at some chosen time interval before the scheduled examination date.

In some embodiments disclosed herein, a patient scheduling component is included. This may include a proactive component that predicts likely patient delays based on information such as the patient's zip code (for outpatients), weather forecasts, roadwork, or so forth; as well as a real time estimated time of arrival (ETA) estimator that tracks patient location by GPS or other information.

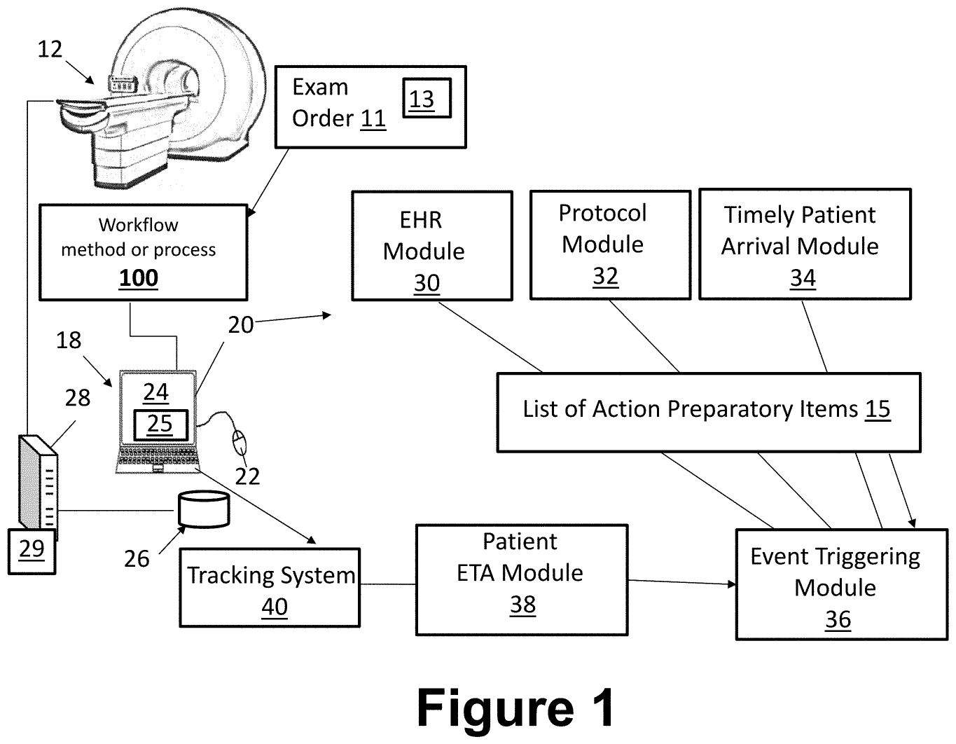

With reference to FIG. 1, an illustrative apparatus 10 for assessing preparedness for a medical examination is shown. The apparatus 10 is used in conjunction with a medical examination order 11 (such as a requisition form for an imaging protocol) which orders an imaging examination using an image acquisition device (also referred to as an imaging device) 12. The medical examination order 11 can include scheduled examination information 13, which can include, for example, a patient identifier (e.g., a PMRN), a scheduled date of the examination, an imaging modality (if the examination is a medical imaging examination), and other information. The apparatus 10 can be used to generate one or more preparatory action items 15 to be performed in preparation for the medical examination reflected in the medical examination order 11.

The image acquisition device 12 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 12 may also be a hybrid imaging device such as a PET/CT or SPECT/CT imaging system. It should be noted that the examination order 11 typically does not identify a specific imaging device that is to perform the imaging examination, but usually only identifies the imaging modality. Depending upon the particulars of imaging examination being ordered, any available imaging device of that modality may be suitably used to perform the imaging examination, or some subset thereof. (As an example of the latter, certain CT examinations may require a large bore, so that only those CT scanners with appropriate bore size can be used).

FIG. 1 also shows an electronic processing device 18, such as a workstation computer, or more generally a computer. Alternatively, the electronic processing device 18 can be embodied as a server computer or a plurality of server computers, e.g. interconnected to form a server cluster, cloud computing resource, or so forth. The workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18. The display device 24 can also be configured to display a graphical user interface (GUI) 25.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to assessing preparedness for a medical examination.

The apparatus 10 also includes, or is otherwise in operable communication with, a database 28 storing patient information 29 contained in medical records of patients. The database 28 can be any suitable database, including a Radiology Information System (RIS) database, a Picture Archiving and Communication System (PACS) database, an Electronic Medical Records (EMR) database, an Electronic Health Records (EHR) database, various combinations thereof, and so forth. As used herein, the database 28 refers primarily to an EHR database. Alternatively, the database 28 can be implemented in the non-transitory medium or media 26. The workstation 18 can be used to access the patient information 29 in the database 28.

As shown in FIG. 1, the at least one electronic processor 20 is programmed to implement a plurality of modules to assess patient preparedness for a medical examination based on information contained in the examination order 11 and contained in the patient information database(s) 28. A medical record (i.e., EHR) module 30 is configured to interface with the EHR 28. The EHR module 30 is configured to parse through the patient records in the EHR 28 to find instances of hospital stays, doctor visits, practitioner notes, medical reports (e.g., radiology reports, pathology reports, etc.), and scanned medical record pages from other medical facilities. The EHR module 30 is then programmed to extract (e.g., by natural language processing algorithms and/or machine learning algorithms) mentions of: (i) foreign objects, such as implanted medical devices (which can present a safety concern for MRI exams and could potentially affect the quality of the imaging session); (ii) imaging contrast agent allergies (in which a patient should either be pre-medicated with anti-histamines or assigned to an imaging procedure that does not use a contrast agent); (iii) prior imaging studies of the patient (which are important for follow-up procedures, as well as ensuring the patient does not receive multiple contrast doses in quick succession); (iv) medications (some drugs can enhance allergic reactions to contrast or increase the likelihood of an allergic reaction taking place. In addition, contrast agents tend to prolong clotting time; and exasperating effects of anti-coagulant drugs, and may have to be adjusted prior to contrast administration); (v) claustrophobia (to ensure patient has necessary medications at the next visit); (vi) data related to prior adverse events (e.g., due to contrast reaction, contrast extravasation, claustrophobia or other cause, it is important to know if the patient had a negative experience in order to avoid a repeat occurrence); (vii) a need for translation services for the patient; (viii) patients with disabilities (e.g., requiring wheelchairs) to allow extra preparation time; and (ix) and occupation of the patient (e.g., certain occupations are more likely to be claustrophobic (i.e., military), while patients in occupations such as metalworking or welding or those in police work may require an extra CT scan prior to receiving MRI scan to check on metal shards or shrapnel pieces).

The EHR module 30 may employ pre-constructed search patterns that are run against natural language textual content stored in the EHR database 28. For example, the pre-constructed search patterns may search for specific words such as “implant”, “pacemaker”, or so forth; combinations of words such as occupation NEAR3 welder; word roots or combinations including word roots, such as (occupation NEAR3 metal*) where “metal*” matches any word beginning with “metal”; and/or so forth. In the case of handwritten content such as physician notes, it is contemplated to apply optical character recognition (OCR) to extract natural language text from the handwritten notes, against which the pre-constructed search patterns are applied. The EHR module 30 may also be programmed to read structured patient information content when documents with the suitable structure are available. For example, if the hospital uses a standard patient admissions form with structured data entry fields, then the EHR module 30 can be programmed to automatically read content of the fields based on the known document structure. The EHR module 30 may also be programmed to recognize standard clinical codes, such as International Classification of Diseases (ICD) codes, and leverage the information defined by those codes. For example, ICD-10 code F40.240 indicates a claustrophobia diagnosis, and hence a search for code F40.240 can be used to identify a patient having claustrophobia. These are merely examples, and other approaches can be employed by the EHR module 30 for extracting salient information from the EHR database 28.

A protocol module 32 is configured to identify patients who need to be contacted, notified, and reminded of preparatory actions needed to prepare for a medical examination. In one example, some patients may be required to perform (or schedule to have performed) some preparation work, such as fasting or an enema. In another example, blood work may be scheduled for the patient.

A timely patient arrival module 34 is configured to predict a likelihood of a late arriving patient or a “no show” patient using predictive algorithms and patient information from the database 28 including patient past history of appointment attendance, level of patient engagement, the nature of the exam, the patient's zip-code, and date and time of the examination. The timely patient arrival module 34 can also use other data, such as weather conditions, sporting events (e.g., causing traffic delays), construction zones, strikes, and any seasonal factors that could affect traffic or public transportation delays at the time of patient's examination.

The outputs of the EHR, protocol, and arrival modules 30, 32, 34 output to an event triggering module 36 that is configured to trigger alerts to particular patients and/or specific medical personnel or groups of medical personnel. In one example, for patients who are indicated to have a higher likelihood of being late or missing their appointments, the event triggering module 36 will be activated to engage the patient. For patients whose appointment will be affected by sporting events, concerts, etc., a proactive message can be dispatched through the event triggering module 36. In addition, the event triggering module 36 can trigger alerts if the EHR module 30 finds pertinent information within a patient's medical record (e.g., the patient may be sent a reminder to bring his sedative for the exam or take an anti-histamine for a contrast allergy or bring a safety card for a medical implant). In another example, the event triggering module 36 can trigger alerts if the protocol module 32 determines that a patient preparation step is required (e.g., fasting). In a further example, the event triggering module 36 can trigger alerts if the timely patient arrival module 34 determines that patient's timely arrival is suspect. In each case, a patient may be sent reminder text messages or phone calls. In addition, the event triggering module 36 can send reminders to, for example, a modality manager, administrative personnel, technologist personnel, and nursing staff (when appropriate) of any potential issues, so that proactive measures are taken to ensure patient safety and comfort, and any workflow challenges associated with patient needs for interpretation or transportation services or risks associated with patient tardiness are mitigated.

In some embodiments, the triggering module 36 is also programmed to automatically or semi-automatically schedule tasks that address certain preparatory action items. For example, the triggering module 36 may send the patient a proposed date for drawing blood for a required blood test. Upon receiving assent from the patient (or, alternatively, receiving a counter-proposed date from the patient), the triggering module 36 then accesses an electronic calendar of the hematology laboratory to place the blood draw on the schedule. Similarly, if an ocular CT screening is a preparatory action item then the triggering module 36 can place an appropriate CT scan examination onto the CT schedule. As another example, if a preparatory action item is to provide a translator, then the triggering module 36 may access a hospital staff scheduling database and place a request for an interpreter for the patient's native language at the scheduled time of the imaging examination.

A patient expected-time-of-arrival (ETA) module 38 is configured to track a patient's progress in arriving to a medical facility for the medical examination. This ETA module 38 is configured to operate a network 40 (e.g., a GPS, Wi-Fi, or Internet Network) is used as a tracking system to track patients' cell phones to assess in real time the likelihood of a patient arriving to the facility early, on time, or late for the appointment.

The apparatus 10 is configured as described above to perform a method or process 100 for improving preparedness for a medical examination by identifying preparatory action items 15 and pushing reminders and alerts. The non-transitory storage medium 26 stores instructions which are readable and executable by the at least one electronic processor 20 to perform disclosed operations including performing the method or process 100 for assessing preparedness for a medical examination. In some examples, the method 100 may be performed at least in part by cloud processing.

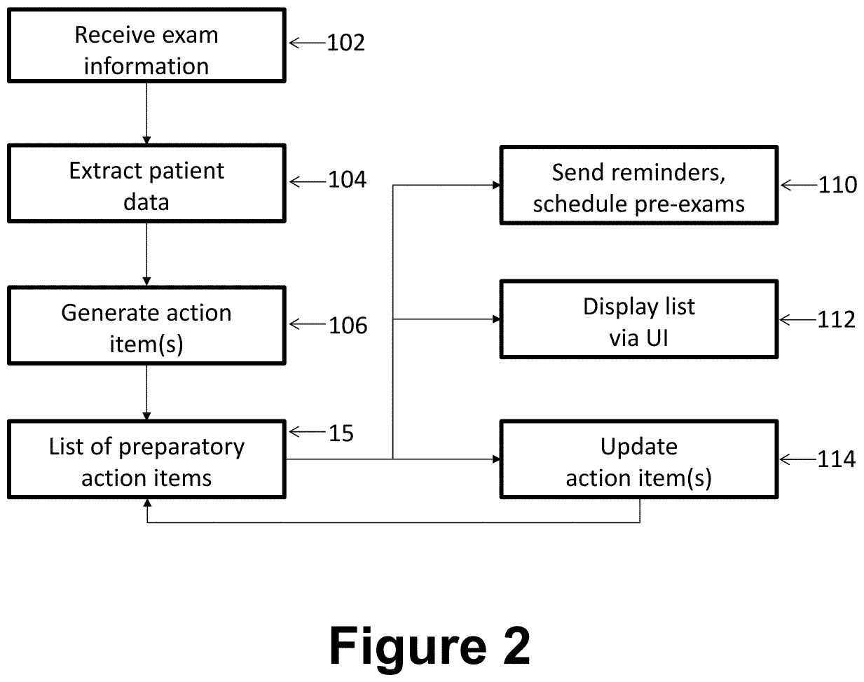

Referring now to FIG. 2, an illustrative embodiment of method or process 100 is diagrammatically shown as a flowchart. At an operation 102, the at least one electronic processor 20 is programmed to receive the scheduled examination information 13 from the examination work order 11. The received information 13 can include, for example, a patient identifier (e.g., a PMRN), a scheduled date of the examination, and an imaging modality if the examination is a medical imaging examination. In some examples, the at least one electronic processor 20 is programmed to provide the GUI 25 on the display device 24 via which a user schedules the medical examination work order 11. Alternatively, the scheduled examination information 11 can be retrieved from the non-transitory computer readable medium 26 of the workstation 18.

At an operation 104, the at least one electronic processor 20 is programmed to extract the patient information 29 from one or more records of the patient (e.g., stored in the EHR database 28 and accessed by the EHR module 30) based on the scheduled imaging examination information 13. For example, the patient information 29 for a particular patient can be found based on the patient medical record number contained in the scheduled examination information 13.

At 106, the at least one electronic processor 20 is programmed to generate the list of one or more preparatory action items 15 to be performed in preparation for the medical examination based on the patient information 29 and the scheduled imaging examination information 13. The list of preparatory action item(s) 15 can be variously used. For example, at 110, reminders may be sent out by the triggering module 36, and/or action items 15 can be automatically scheduled. In another example, at 112, the action items 15 can be displayed on the display device 24. For example, clerical staff at the imaging laboratory may pull up the list of preparatory action items 15 for all patients scheduled for the next day (or two days ahead, or some other time interval) to ensure that all patients are ready for tomorrow's examinations. In a further example of operation 112, the preparatory action items 15 can be stored in the non-transitory computer readable medium 26 of the workstation 18, for later retrieval by a medical professional. By way of illustration, when a patient is checked into the imaging laboratory, the list of preparatory items 15 for that patient can be brought up and verified to be completed.

With continuing reference to FIG. 2, in some embodiments, at 114 the preparatory action items 15 are dynamically updated by the apparatus 10. For example, the apparatus 10 can monitor the EHR database 28 to detect whether the patient has had required bloodwork. If the required bloodwork results are identified in the EHR database 28 then that preparatory action item can be removed from the list of action items 15. Similarly, if an action item 15 is to schedule a translator, then the apparatus 10 can monitor a staff scheduling database to verify a translator (of the appropriate language) has been scheduled to the imaging laboratory at the scheduled examination date—if so, then the translator preparatory action item may be removed.

Several examples of the relationship between the patient information 29 and the scheduled examination information 13, and the resulting generated action items 15 will be discussed in more detail below. In a first example, the at least one electronic processor 20 is programmed to identify a combination of an imaging modality being magnetic resonance imaging (MRI) from the scheduled examination information 13, and the patient information 29 including a presence of a medical implant in the patient. In response, an MRI classification of the medical implant is determined from the extracted patient information 29. Then, unless the MRI classification of the medical implant is MRI-safe, a preparatory action item 15 is generated in accordance with the MRI classification of the medical implant. That is, if the implant is determined to be safe for MRI (e.g., the implant does not include metal), then the action item 15 can include one or more of providing a cardiac team on standby, imaging the patient with a magnetic field less than 3 Tesla, and so forth. On the other hand, it the implant is classified as not being MRI-safe, then preparatory action item 15 can include rescheduling the patient for a different imaging modality.

In another example, the at least one electronic processor 20 is programmed to identify a combination of the imaging modality being magnetic resonance imaging (MRI) from the scheduled examination information 13, and the patient information 29 including an occupation of the patient associated with metalworking (e.g., a welder). In response, the generated preparatory action item 15 can include scheduling an ocular CT screening for the patient to detect of metallic objects in the eyes of the patient.

In a further example, the at least one electronic processor 20 is programmed to identify a combination of a native language of the patient and a prior use of a translator for the patient from the patient information 29. The corresponding generated action item 15 can include scheduling availability of a native language translator for the patient.

In yet another example, the at least one electronic processor 20 is programmed to identify a patient mobility limitation of the patient (e.g., whether the patient requires a wheelchair, a walking guide if the patient is blind, a gurney if the patient is bedridden, etc.) from the patient information 29. The corresponding generated action item 15 can include scheduling availability of a transport support for the patient.

In another example, the at least one electronic processor 20 is programmed to identify a prior use of non-emergency medical transportation for the patient from the patient information 29. The corresponding generated action item 15 can include scheduling non-emergency medical transportation for the patient.

In a further example, the at least one electronic processor 20 is programmed to identify a combination of a contrast agent to be used in the medical imaging examination from the scheduled imaging examination information 13, and an allergy to the contrast agent in the patient information 29. In response, the generated preparatory action item 15 can include revising the medical imaging examination to use a different contrast agent or no contrast agent.

In another example, the at least one electronic processor 20 is programmed to identify required pre-examination bloodwork for the medical imaging examination from the scheduled imaging examination information and no indication of the required pre-examination bloodwork from the patient information 29. The corresponding generated action item 15 can include scheduling the pre-examination bloodwork.

In yet another example, the at least one electronic processor 20 is programmed to identify pre-examination fasting for the medical imaging examination from the scheduled imaging examination information 13. The corresponding generated action item 15 can include scheduling an electronic reminder notification to the patient of the required pre-examination fasting.

In another example, the at least one electronic processor 20 is programmed to identify the imaging modality being a nuclear imaging modality and identifying a radiation dose to be delivered to the patient by the medical imaging examination from the scheduled imaging examination information 13, and computing a prior cumulative radiation dose received by the patient from the patient information 29. The at least one electronic processor 20 then determines whether the sum of the prior cumulative radiation dose and the radiation dose to be delivered exceeds a maximum permissible cumulative radiation dose. The corresponding generated action item 15 can include either adjusting the radiation dose to be delivered in accordance with the maximum permissible cumulative radiation dose, or revising the medical imaging examination to use a non-nuclear imaging modality. These are merely illustrative examples, and are not intended to limit the scope of potential action items 15.

In some embodiments, the at least one electronic processor 20 is programmed to maintain status information for the action items 15. The status information can include, for example, whether the patient performed the required fasting, or whether the pre-examination blood work has been obtained, and so forth. The at least one electronic processor 20 is programmed to automatically updating the one or more action items 15 based on the status information.

In other embodiments, the at least one electronic processor 20 is programmed to transmit one or more reminders to the patient regarding the action items 15. The reminders can be text messages, voicemails, emails, and so forth, and can be reminders to perform pre-examination fasting, to inform the patient of a potential traffic delay, and so forth. The at least one electronic processor 20 (e.g., using the protocol module 32 and the event triggering module 36) is programmed to automatically updating the one or more action items 15 based on responses by the patient to the reminders (e.g., a text message indicating the patient performed any required fasting, whether the patient is going to be late for the examination based on traffic information, and so forth).

In further embodiments, the at least one electronic processor 20 (e.g., using the protocol module 32, the timely patient arrival module 34, the event triggering module 36, and the patient ETA module 38) is programmed to receive movement regarding transportation of the patient, including a location of the patient, tracking movement of the patient with GPS, weather forecast, roadwork, and so forth. Using this information, the at least one electronic processor 20 is programmed to predict changes to the action items 15.

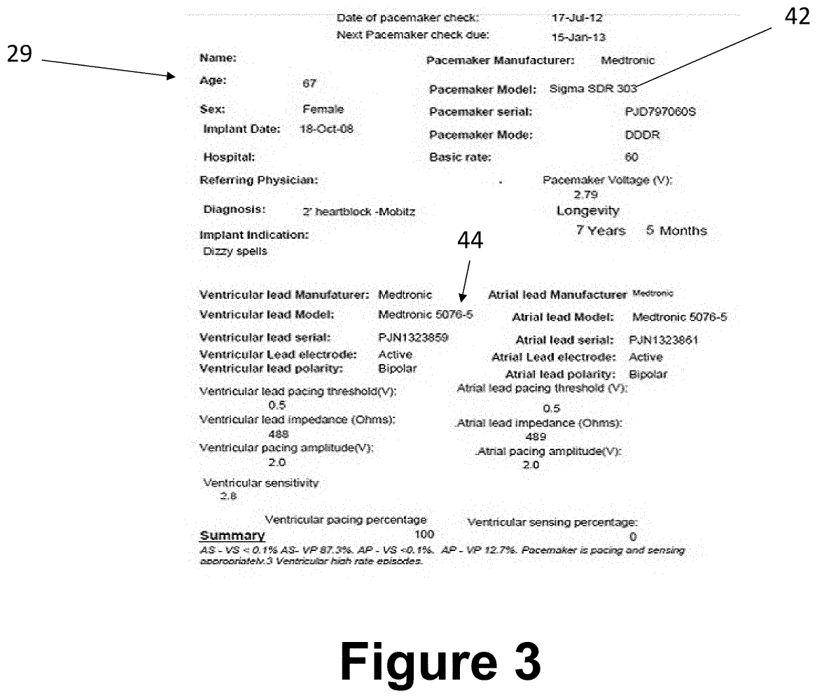

FIG. 3 shows an example of the generation of an action item 15. In this example, a patient calls to schedule an appointment for MRI scan. The apparatus 10 automatically mines patient records in the EHR database 28 for mentions of medical devices, and can return a finding that the patient has a MRI-conditional pacemaker with leads, as indicated by reference characters 42 and 44. The apparatus 10 can, for example, schedule the appointment to an appropriate 1.5T imaging device. Additionally, the apparatus 10 can send alerts to a modality manager to arrange for the presence of cardiology staff on site to ensure patient safety.

Additionally or alternatively, the apparatus 10 can mine the patient's information 29 in the EHR database 28 for the patient's occupation, which can return a finding that the patient is a retired welder. The apparatus 10 can send a message to the patient asking if an ocular CT had been performed in the past and if not, schedules an ocular CT scan prior to patient's MRI scan.

Additionally or alternatively, the apparatus 10 can review the patient information 29 and find that the patient has a contrast allergy for the contrast agent used during the scheduled scan. The apparatus 10 can send an alert to the modality manager/charge tech/referring physician, and send a message to the patient to take anti-histamine medication 24 hours prior to the scan.

Additionally or alternatively, the apparatus 10 can review the examination order 11 and find that the patient does not have the necessary bloodwork done. The apparatus 10 can send a reminder to the patient to get bloodwork done prior to the scan.

Additionally or alternatively, the apparatus 10 can again review the patient information 29, and informs modality manager, technologist, and nurse that the patient does not have the required bloodwork, and can also send a message to the patient to arrive early to have sufficient time to have the bloodwork done on site.

Additionally or alternatively, the apparatus 10 can discover that patient's previous MR scan was shortened due to claustrophobia, generate an alert the modality manager and the referring physician, and send a message to the patient to bring a sedative to the appointment.

Additionally or alternatively, the apparatus 10 can find the need for interpreter services in patient's records, and send an alert to the modality manager and administrative staff informing them of the need to arrange for interpreter services.

Additionally or alternatively, the apparatus 10 can reviews the work order 11 or the protocoled study and messages the patient with required preparation instructions—such as fasting or enema, etc.

Additionally or alternatively, the apparatus 10 can identify patient's likelihood of arriving late or not showing up for the appointment, and sends alerts and reminders to the patient regarding the appointment, asking to confirm the intention to attend the appointment.

Additionally or alternatively, the apparatus 10 can the network 40 to track patient progress during transport to the imaging facility for their appointments, and can provide ETA information to the modality manager, technologists, administrative staff and sends alerts if the patient is running significantly late or are likely to arrive significantly early.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An apparatus, comprising:
at least one electronic processor programmed to:
receive scheduled imaging examination information for a medical imaging examination which the patient is to undergo including at least a patient identifier, a scheduled date of the medical imaging examination, and an imaging modality of the medical imaging examination;
extract patient information from one or more medical records of the patient based on the scheduled imaging examination information; and
generate one or more preparatory action items to be performed in preparation for the medical imaging examination based on the patient information and the scheduled imaging examination information.

2. The apparatus of claim 1, wherein the at least one electronic processor is programmed to receive the scheduled imaging examination information by operations including providing a user interface via which a user schedules the medical imaging examination, and is further programmed to generate the one or more preparatory action items from the patient information and the schedule examination information by at least one of:
automatically scheduling at least one of the one or more preparatory action items during a portion of the examination;
displaying the one or more preparatory action items on a display device;
storing the one or more preparatory action items for retrieval by a medical personnel.

3. The apparatus of claim 1, wherein the at least one electronic processor is programmed to generate the one or more preparatory action items by operations including:
identifying a combination of the imaging modality being magnetic resonance imaging (MRI) and the patient information including a presence of a medical implant in the patient; and
in response determining an MRI classification of the medical implant from the extracted patient information; and
unless the MRI classification of the medical implant is MRI-safe, generating a preparatory action item in accordance with the MRI classification of the medical implant.

4. The apparatus of claim 1, wherein the at least one electronic processor is programmed to generate the one or more preparatory action items by operations including:
identifying a combination of the imaging modality being magnetic resonance imaging (MRI) and the patient information including an occupation of the patient which is associated with metalworking; and
in response, generating a preparatory action item comprising scheduling an ocular computed tomography (CT) screening for the patient.

5. The apparatus of claim 1, wherein the at least one electronic processor is programmed to generate the one or more preparatory action items by operations including:
identifying a native language of the patient and a prior use of a translator for the patient in the patient information; and
in response, generating a preparatory action item comprising scheduling availability of a native language translator for the patient.

6. The apparatus of claim 1, wherein the at least one electronic processor is programmed to generate the one or more preparatory action items by operations including:
identifying a patient mobility limitation of the patient in the patient information; and
in response, generating a preparatory action item comprising scheduling availability of transport support for the patient in accordance with the identified patient mobility limitation.

7. The apparatus of claim 1, wherein the at least one electronic processor is programmed to generate the one or more preparatory action items by operations including:
identifying a prior use of non-emergency medical transportation for the patient in the patient information; and
in response, generating a preparatory action item comprising scheduling non-emergency medical transportation for the patient.

8. The apparatus of claim 1, wherein the at least one electronic processor is programmed to generate the one or more preparatory action items by operations including:
identifying a contrast agent to be used in the medical imaging examination from the scheduled imaging examination information and an allergy to the contrast agent in the patient information; and
in response, generating a preparatory action item comprising revising the medical imaging examination to use a different contrast agent or no contrast agent.

9. The apparatus of claim 1, wherein the at least one electronic processor is programmed to generate the one or more preparatory action items by operations including:
identifying required pre-examination bloodwork for the medical imaging examination from the scheduled imaging examination information and no indication of the required pre-examination bloodwork in the patient information; and
in response, generating a preparatory action item comprising scheduling the pre-examination bloodwork.

10. The apparatus of claim 1, wherein the at least one electronic processor is programmed to generate the one or more preparatory action items by operations including:
identifying required pre-examination fasting for the medical imaging examination from the scheduled imaging examination information; and
in response, generating a preparatory action item comprising scheduling an electronic reminder notification to the patient of the required pre-examination fasting.

11. The apparatus of claim 1, wherein the at least one electronic processor is programmed to generate the one or more preparatory action items by operations including:
identifying the imaging modality being a nuclear imaging modality and identifying a radiation dose to be delivered to the patient by the medical imaging examination from the scheduled imaging examination information;
computing a prior cumulative radiation dose received by the patient from the patient information;
determining the sum of the prior cumulative radiation dose and the radiation dose to be delivered exceeds a maximum permissible cumulative radiation dose; and
in response, generating a preparatory action item comprising one of (i) adjusting the radiation dose to be delivered in accordance with the maximum permissible cumulative radiation dose or (ii) revising the medical imaging examination to use a non-nuclear imaging modality.

12. The apparatus of claim 1, wherein the at least one electronic processor is programmed to:
maintain status information for the one or more action items; and
automatically update the one or more action items based on the status information.

13. The apparatus of claim 1, wherein the at least one electronic processor is programmed to:
transmit one or more reminders to the patient regarding the one or more action items.

14. The apparatus of claim 13, wherein the at least one electronic processor is programmed to:
automatically update the one or more action items based on responses from the patient to the one or more reminders.

15. The apparatus of claim 1, wherein the at least one electronic processor is programmed to:
receive time change information including one or more of a location of the patient, weather forecast, and roadwork; and
predict changes to the one or more action items based on the time change information.

16. The apparatus of claim 15, wherein the at least one electronic processor is further programmed to:
track a movement of the patient by GPS; and
predict changes to the one or more action items based on the movement.

17. A non-transitory computer readable medium storing instructions executable by at least one electronic processor to perform a method of generating one or more preparation action items to be performed in preparation for a medical examination which a patient is to undergo, the method comprising:
receiving scheduled examination information for the medical examination including at least a patient identifier, a scheduled date of the medical examination, and an imaging modality of the medical imaging examination;
extracting patient information from one or more medical records of the patient based on the scheduled examination information;
generating one or more preparatory action items to be performed in preparation for the medical examination based on the patient information and the scheduled examination information;
transmitting one or more reminders to the patient regarding the one or more action items; and
automatically updating the one or more action items based on responses from the patient to the one or more reminders.

18. The non-transitory computer readable medium of claim 17, wherein the method further includes:
receiving the scheduled examination information by operations including providing a user interface via which a user schedules the medical examination; and
generating the one or more preparatory action items from the patient information and the schedule examination information by at least one of:
automatically scheduling at least one of the one or more preparatory action items during a portion of the examination;
displaying the one or more preparatory action items on a display device;
storing the one or more preparatory action items for retrieval by a medical personnel.

19. The non-transitory computer readable medium of claim 17, wherein the method further includes:
receiving time change information including one or more of a location of the patient, a movement of the patient, a weather forecast, and roadwork; and
predict changes to the one or more action items based on the time change information.

20. A method of generating one or more preparation action items to be performed in preparation for a medical examination which a patient is to undergo, the method comprising:
receiving scheduled imaging examination information for the medical imaging examination including at least a patient identifier, a scheduled date of the medical examination, and an imaging modality of the medical examination;
extracting patient information from one or more medical records of the patient based on the scheduled examination information;
generating one or more preparatory action items to be performed in preparation for the medical imaging examination based on the patient information and the scheduled examination information;
receiving time change information including one or more of a location of the patient, a movement of the patient, a weather forecast, and roadwork; and
predict changes to the one or more action items based on the time change information.
